(19) United States
(12) Patent Application Publication
(10) Pub. No.: US 2014/0336599 A1
(43) Pub. Date: Nov. 13, 2014

(54) SUCTION MANIFOLD ASSEMBLY

(71) Applicant: Thermedx, LLC, Solon, OH (US)

(72) Inventors: Nilesh Rajnikant Patel, Solon, OH (US); Douglas L. Carr, Chardon, OH (US)

(73) Assignee: Thermedx, LLC, Solon, OH (US)

(21) Appl. No.: 13/892,629

(22) Filed: May 13, 2013

(51) Int. Cl. A61M 1/00

(52) U.S. Cl. CPC A61M 1/0023; A61M 1/0056
USPC 604/319

(57) ABSTRACT

A suction manifold assembly for a fluid management system comprised of a suction manifold and a mounting member. The suction manifold includes a single outlet port and a plurality of inlet ports, including a removable inlet port having a tissue collection device for collecting excised tissue. The suction manifold assembly enables convenient and precise adjustment of suction levels of inputs into fluid collection containers, integrates tissue collection with suction operations, and provides strain relief to tubing thereby allowing accurate fluid deficit monitoring operations.

### FIELD OF THE INVENTION

The present invention relates generally to a fluid manifold, and more particularly to a suction manifold assembly adapted for use with a fluid management system.

### BACKGROUND OF THE INVENTION

A fluid management system may be used to manage fluid delivery in connection with several different fluid delivery functions, including, but not limited to, irrigation, and distention of a body cavity. These fluid delivery functions may be carried out in connection with a wide variety of medical procedures, including, but not limited to, hysteroscopy, cystoscopy, ureteroscopy, laparoscopy, and arthroscopy. These medical procedures are associated with several different medical disciplines, such as, gynecology, urology, orthopedics, and general surgery. An exemplary fluid management system is described in U.S. patent application Ser. No. 12/720,475 (filed Mar. 9, 2010), published on Sep. 9, 2010 as U.S. Patent Application Publication No. US2010/0228222 A1, the disclosure of which is fully incorporated herein by reference.

For certain medical procedures, the fluid management system uses suction to collect fluids in one or more fluid collection containers (e.g., suction canisters) in association with a device, such as a scope or instrument having a fluid outflow port (e.g., a hysteroscope), an under-buttocks drape having a suction port, and a floor fluid suction device. Accordingly, fluid may be collected at a surgical site, under a patient during surgery, and from an operating room floor. The fluid management system may also provide a fluid deficit monitoring function for determining the amount of fluid that has been delivered by the fluid management system, but has not been returned to the fluid collection containers.

In situations where a plurality of devices are used to collect fluid in the fluid collection containers during a single procedure, system setup can be complex. In this regard, tubing must be configured between each device and an associated fluid collection container. If there is only a single fluid collection container with a single input port, then it may become necessary to connect and reconnect a selected device to the single fluid collection container.

Moreover, different suction levels may be desirable for different devices, depending upon the application. Suction levels typically range from 50 mmHg to 500 mmHg. Prior art systems do not allow for convenient adjustment of suction levels for each device connected to the fluid collection container.

There is a need to simplify the setup for configuring tubing used by the fluid management system to carry out fluid suction operations; enable convenient and precise adjustment of suction levels of inputs into fluid collection containers to ensure proper operation of all surgical instrumentation; integrate tissue collection with suction operations; and provide strain relief to tubing in order to ensure vibration and/or movement of the tubing does not adversely affect the accuracy of fluid deficit monitoring operations.

The present invention provides a suction manifold assembly that addresses the foregoing deficiencies of the prior art.

### SUMMARY OF THE INVENTION

In accordance with the present invention, there is provided a suction manifold assembly that includes a suction manifold comprising (a) a body defining an inner region; (b) a plurality of inlet ports, each inlet port having a passageway in fluid communication with the inner region and a fitting adapted for connection with a hose, wherein said plurality of inlet ports may include a removable inlet port having a tissue collection device; and (c) an outlet port having a passageway in fluid communication with the inner region and a fitting adapted for connection with a hose.

In accordance with another aspect of the present invention, there is provided a suction manifold for receiving fluid from a plurality of fluid sources, said suction manifold comprising: (a) a body defining an inner region; (b) a plurality of inlet ports, each inlet port having a passageway in fluid communication with the inner region and a fitting adapted for connection with a hose, wherein said plurality of inlet ports may include a removable inlet port having a tissue collection device for collecting excised tissue, said tissue collection device allowing fluid to pass therethrough; and (c) an outlet port having a passageway in fluid communication with the inner region and a fitting adapted for connection with a hose.

An advantage of the present invention is the provision of a suction manifold assembly that simplifies configuration of tubing used by the fluid management system to carry out fluid suction operations.

Another advantage of the present invention is the provision of a suction manifold assembly that enables convenient and precise adjustment of suction levels of inputs into fluid collection containers.

A still further advantage of the present invention is the provision of a suction manifold assembly that integrates tissue collection with fluid suction operations.

Yet another advantage of the present invention is the provision of a suction manifold assembly that provides tubing strain relief to ensure vibration and/or movement of the tubing does not adversely affect accuracy of fluid deficit monitoring operations.

These and other advantages will become apparent from the following description of illustrated embodiments taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a schematic view of a suction manifold assembly, comprised of a suction manifold and mounting member, according to an embodiment of the present invention;

FIG. 2 is a cross-sectional view of the suction manifold assembly of FIG. 1;

FIG. 3 is an exploded perspective view of the suction manifold assembly of FIG. 1;

FIG. 4 shows a mounting member according to another embodiment;

FIG. 5 shows a suction manifold according to an alternative embodiment.

### DETAILED DESCRIPTION OF THE INVENTION

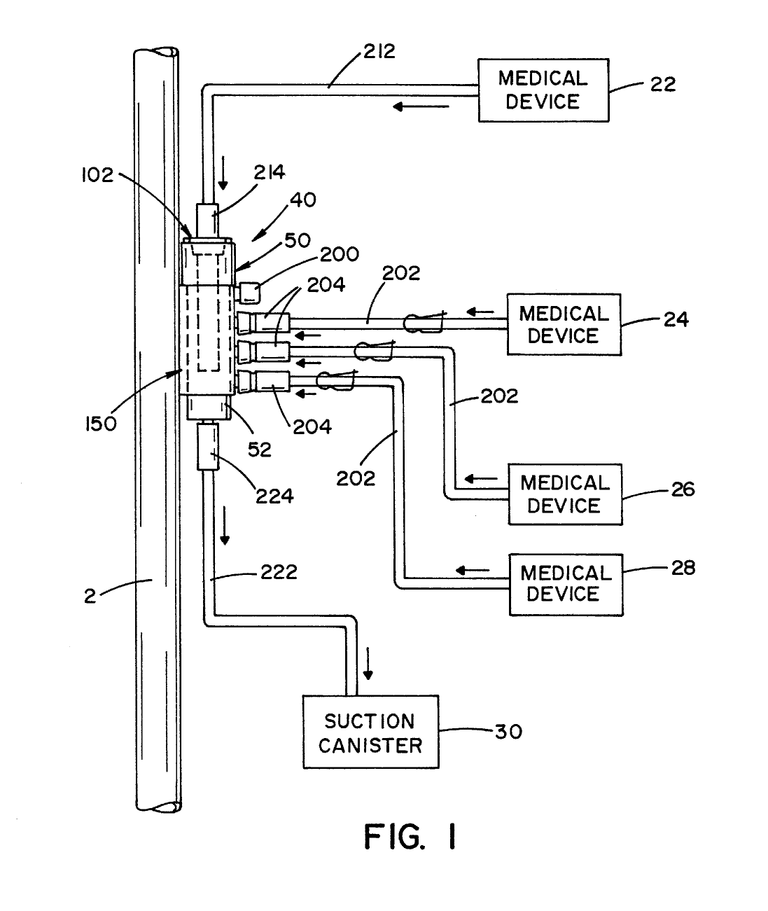

Referring now to the drawings wherein the showings are for the purposes of illustrating a preferred embodiment of the invention only and not for the purposes of limiting same, FIG. 1 shows a schematic view of a suction manifold assembly 40, according to a first embodiment. Suction manifold assembly 40 is generally comprised of a suction manifold 50 and a mounting member 150. In FIG. 1, mounting member 150 is shown mounted to a support structure 2 taking the form of a cylindrical pole. FIG. 1 also shows suction manifold 50 fluidly connected with medical devices 22, 24, 26 and 28 and a suction canister 30, as will be described in further detail below.

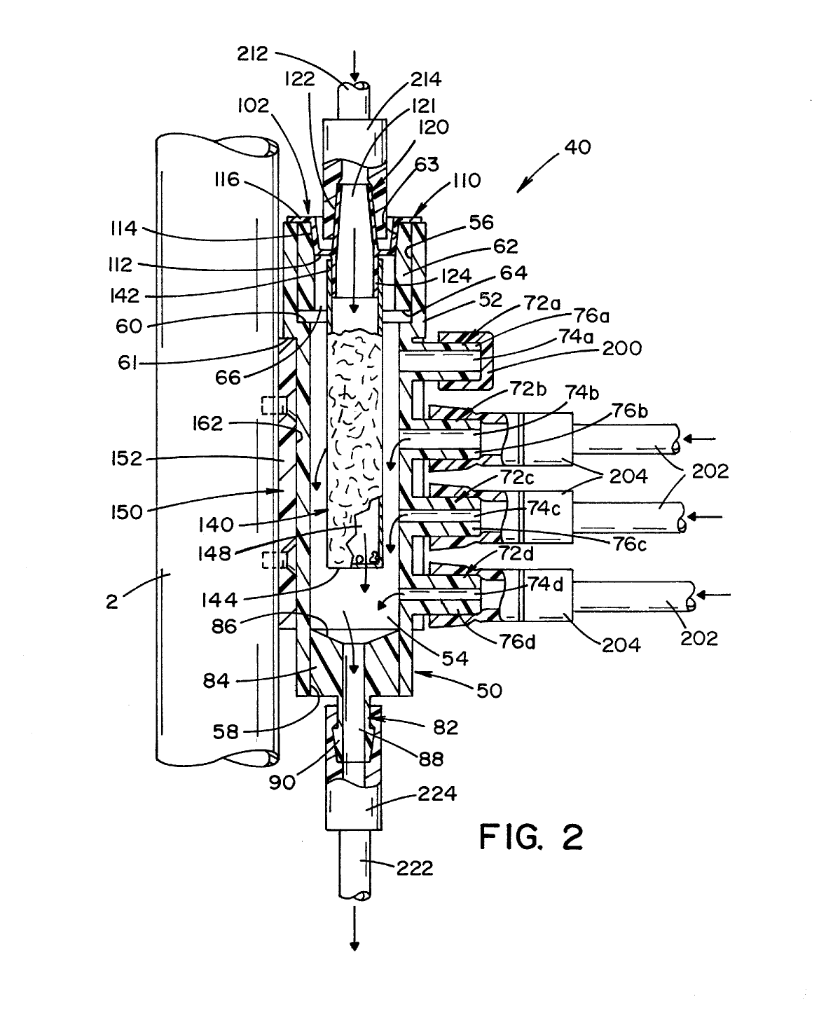

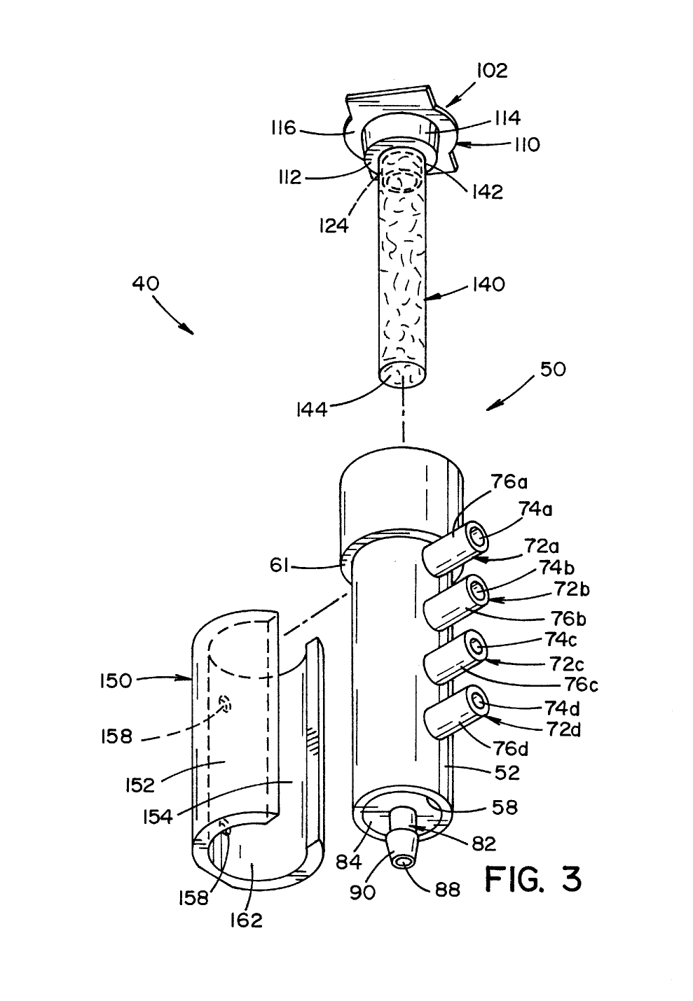

With reference to FIGS. 2 and 3, suction manifold 50 is generally comprised of a cylindrical body 52 that defines an inner region 54; a plurality of inlet ports 72a, 72b, 72c, 72d; a removable inlet port 102; and an outlet port 82. In the illustrated embodiment, cylindrical body 52 has a first opening 56 at one end and a second opening 58 at the opposite end. An inner annular ledge 60 and an outer annular ledge 61 of cylindrical body 52 define a transition between a first section of cylindrical body 52 having larger inner and outer diameters and a second section of cylindrical body 52 having smaller inner and outer diameters. Accordingly, first opening 56 has a larger diameter than second opening 58.

In the illustrated embodiment, a generally cylindrical insert 62 is press fit within first opening 56. Insert 62 defines an opening 63 having a smaller diameter than first opening 56. Insert 62 includes a bottom wall 64 having a hole 66 formed therein. It should be appreciated that insert 62 may alternatively be formed as an integral part of cylindrical body 52.

Removable inlet port 102 is generally comprised of a mounting section 110 and a tubular fitting section 120. Mounting section 110 includes a bottom wall 112, through which tubular fitting section 120 extends, a generally cylindrical side wall 114, and an outward extending flange 116. Tubular fitting section 120 has first and second portions 122, 124 that define a passageway 121 extending therethrough. In the illustrated embodiment, first portion 122 takes the form of a frustoconical-shaped fitting and second portion 124 takes the form of a generally cylindrical-shaped tube section. First portion 122 is dimensioned to mate with a fitting 214 on a hose or tubing 212. It is contemplated that first portion 122 may take the form of an alternative fitting, such as hose fitting, barbed fitting, glue fitting, standard male (Luer) conical lock fitting, female (Luer) conical lock fitting, stepped fitting, threaded fitting, and proprietary or industry-standard quick disconnects, such as cam and groove type disconnects.

A generally cylindrical tissue collection device 140, having an open end 142 and a closed end 144 is attached to second portion 124. In this regard, second portion 124 of tubular fitting section 120 is inserted into open end 142 and affixed thereto with an adhesive or the like. Tissue collection device 140 provides a tissue collection chamber 148 for collecting excised tissue and passing fluid. Tissue collection device 140 is made of a straining element, such as a mesh, a filter material, a screen, or a perforated media.

Opening 63 of insert 62 is dimensioned to receive removable inlet port 102. In this regard, removable inlet port 102 is press fit into opening 63, wherein tissue collection device 40 extends through hole 66 into inner region 54 of cylindrical body 52. Removable inlet port 102 is fully seated when flange 116 of mounting section 110 abuts the top edge of insert 62 and cylindrical body 52. In the illustrated embodiment, side wall 114 of mounting section 110 and a portion of the inner surface of insert 62 are both conically-shaped to facilitate insertion and removal of removable inlet port 102. When removable inlet port 102 is inserted into opening 63, passageway 121 and tissue collection chamber 148 are in fluid communication with inner region 54 of cylindrical body 52. After tissue is collected in tissue collection chamber 148, removable inlet port 102 may be removed from cylindrical body 52 to withdraw the collected tissue for further analysis. Accordingly, tissue collection device 140 may be separated from tubular fitting section 120 by breaking an adhesive bond and emptying the contents of tissue collection chamber 148 into a specimen jar. Alternatively, tissue collection device 140 may be directly deposited into a specimen jar.

In the illustrated embodiment, outlet port 82 is dimensioned to be press fit into second opening 58 of cylindrical body 52. Outlet port includes a cylindrical main body portion 84 and a fitting 90 that is dimensioned to mate with a fitting 224 on a hose or tubing 222 fluidly connected to a suction canister 30. A passageway 88 extends through main body portion 84 and fitting 90. Main body portion 84 has a frustoconical-shaped opening 86 in fluid communication with passageway 88. Passageway 88 and opening 86 are in fluid communication with inner region 54 of cylindrical body 52. In the illustrated embodiment, fitting 90 takes the form of a barbed fitting. However, it is contemplated that alternative types of fittings may be substituted for the barbed fitting. It is also contemplated that outlet port 82 may be integrally formed with cylindrical body 52.

Inlet ports 72a-72d are defined by respective fittings 76a-76d, which are dimensioned to mate with a fitting 204 on a hose or tubing 202, and associated passageways 74a-74d. Each passageway 74a-74d is in fluid communication with inner region 54. It should be appreciated that the inner diameter of passageways 74a-74d may vary. For example, the inner diameters of passageways 74a and 74b are larger than the inner diameters of passageways 74c and 74d, thereby providing different suction levels. In the illustrated embodiment, fittings 76a-76d are shown as hose fittings. However, it is contemplated that one or more of fittings 76a-76d may take alternative forms, including, but not limited to, barbed fitting, glue fitting, standard male (Luer) conical lock fitting, female (Luer) conical lock fitting, stepped fitting, threaded fitting, and proprietary or industry-standard quick disconnects, such as cam and groove type disconnects.

Fittings 76a-76d are shown oriented at 90 degrees to the longitudinal axis of cylindrical body 52. However, it is contemplated that fittings 76a-76d may be oriented at a different angle (e.g., 45 degrees) to the longitudinal axis of cylindrical body 52 in order to facilitate connection with tubing 202. Furthermore, the illustrated embodiment of suction manifold 50 has been shown with four (4) inlet ports 72a-72d. However, it should be appreciated that the number of inlet ports may be fewer or greater that four.

As illustrated in FIG. 1, fluid flow through tubing 202 may be partially or fully restricted by use of a conventional multiple-position pinch clamp. A cap 200 dimensioned to mate with fittings 76a-76d may be provided to cover any unused inlet ports 72a-72d. In the embodiment shown in FIG. 2, cap 200 covers unused inlet port 72a. It is contemplated that cap 200 may be tethered to the inlet port by a cable, cord, string, or the like, in order to facilitate re-use of cap 200. Moreover, it should be appreciated that cap 200 may be replaced by an elastomeric valve (not shown) fitted to an inlet port 72a-72d such that connection of a tubing fitting 204 to an inlet port fitting 76a-76d opens the valve to allow fluid communication with the inlet port 72a-72d.

In FIGS. 1-3, mounting member 150 takes the form of a mounting bracket comprised of a C-shaped body 152 having an elongated slot 154. C-shaped body 152 defines a channel 162 dimensioned to receive cylindrical body 52 of suction manifold 50. Cylindrical body 52 is fully seated within channel 162 when outer annular ledge 61 abuts the top edge of C-shaped body 152. Inlet ports 72a-72d extend through elongated slot 154 for access thereto. One or more holes 158 are formed in C-shaped body 152 to fasten mounting member 150 to a support structure (e.g., a pole or plate).

According to an alternative embodiment of the present invention, an integrated dovetail may be formed on a portion of cylindrical body 52 that is dimensioned to mate with a matching recess formed on mounting member 150. This configuration may allow greater access to inlet ports 72a-72d of suction manifold 50.

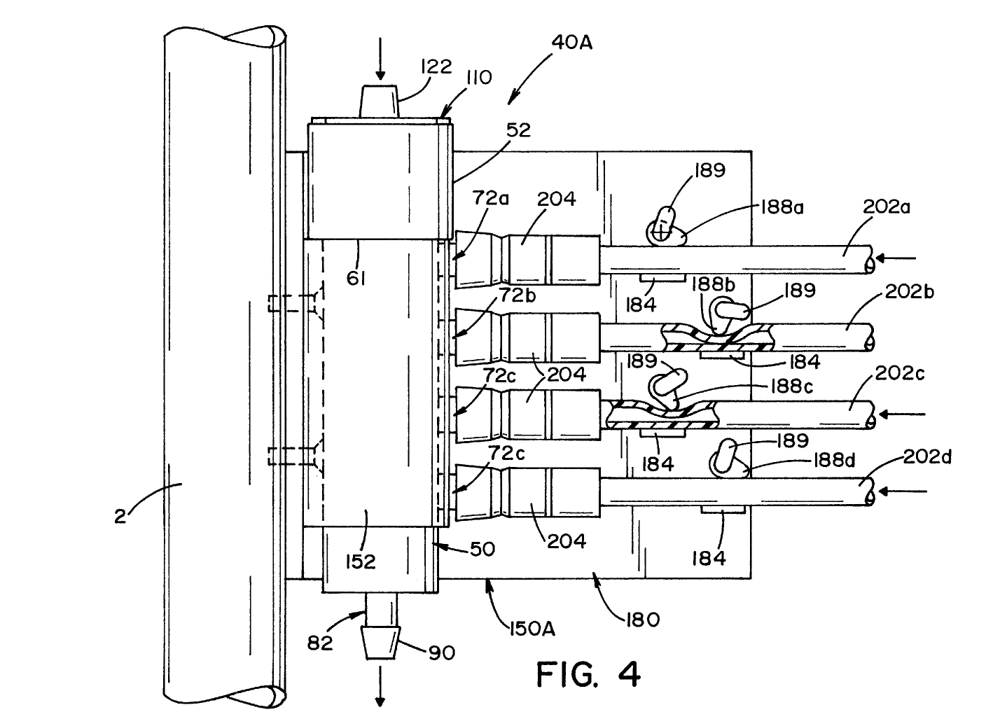

FIG. 4 illustrates a suction manifold assembly 40A according to an alternative embodiment. In this embodiment, suction manifold 50 is unchanged; however, mounting member 150A of suction manifold assembly 40A includes a mounting plate 180. Mounting plate 180 may also be mounted to support structure 2. In the illustrated embodiment, mounting plate 180 includes a flange captured between C-shaped body 152 and support structure 2.

Mounting plate 180 includes a plurality of support members 184 and a plurality of respective rotatable cams 188a-188d having handles 189. Support members 184 are positioned relative to inlet ports 72a-72d such that tubing 202a-202d are supported substantially in alignment with inlet ports 72a-72d. Cams 188a-188d are rotatable to engage with respective tubing 202a-202d (e.g., pinch tubing 202a-202d) and thereby regulate suction level/fluid flow therethrough. In this regard, tubing 202a-202d is captured between a surface of a cam 188a-188d and a support member 184. As illustrated, cams 188a and 188d are shown rotated to a position that does not provide any restriction of suction/fluid flow through tubing 202a and 202d (i.e., an open position). Cam 188c is shown rotated to a position that partially restricts suction/fluid flow through tubing 202c, while cam 188b is shown rotated to a position that fully restricts suction/fluid flow through tubing 202b (i.e., a closed position). It is contemplated that cams 188a-188d may include detents for maintaining a desired position and for repeatable positioning to obtain a desired suction level.

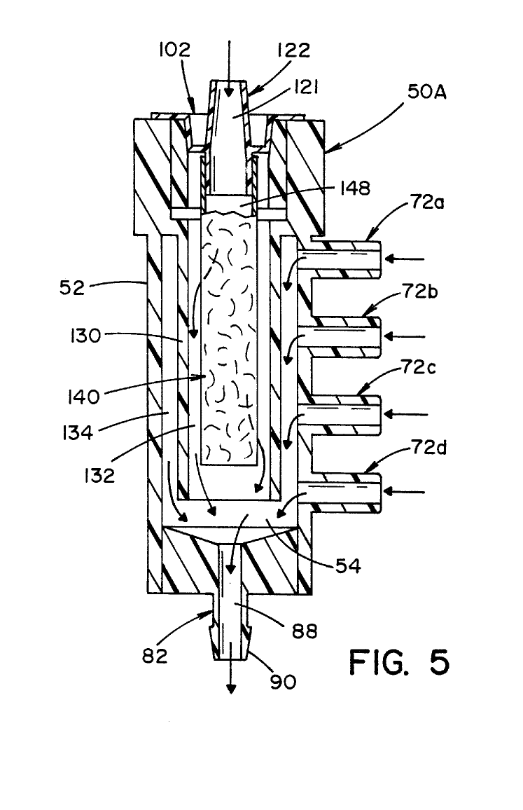

Referring now to FIG. 5, there is shown an alternative embodiment of the suction manifold. Suction manifold 50A includes an inner cylindrical wall 130 that surrounds tissue collection device 140. Accordingly, inner region 54 is divided into an inner annular-shaped chamber 132 that surrounds tissue collection device 140 and an outer annular-shaped chamber 134. Inner cylindrical wall 130 provides separation between fluid received from tissue collection chamber 148 and fluid received from inlet ports 72a-72d.

Suction manifold 50 is preferably constructed of moldable or machinable plastics commonly used in the medical devices art, including, but not limited to, PVC, polycarbonate/acrylic, acetal, nylon, ABS, and like materials, as well as blends thereof. Mounting member 150, 150A is preferably constructed of aluminum, steel, or moldable/machinable plastics commonly used in the medical device arts, and combinations thereof. Cap 200 may be constructed of materials such as vinyl, silicone, rubber, or a rigid plastic that provides adequate seal integrity when covering an inlet port.

It should be appreciated that mounting of suction manifold 50, 50A to a mounting member 150, 150A provides strain relief for tubing 202, 212 and 222 relative to suction canister 30. This strain relief helps prevents vibrations and/or movements of tubing 202, 212 and 222 from causing adverse effects on the accuracy of fluid deficit monitoring operations carried out by a fluid management system.

Referring now to FIG. 1, tubing 212 and 202 are shown connected with medical devices 22-28, while tubing 222 is shown connected with suction container 30. Suction canister 30 is coupled to a suction or vacuum source (not shown), such as those commonly found in a surgical suite. It is contemplated that suction manifold 50 may be used as a disposable device. Therefore, suction manifold 50 may be temporarily attached to a fluid management system by fixing mounting member 150, 150A to a support structure (e.g., pole) of the fluid management system, and inserting suction manifold 50 into channel 162 of mounting member 150 (see FIGS. 1, 2 and 4).

As indicated above, inlet port 102 is removable from cylindrical body 52 of suction manifold 50 to allow for analysis of body tissue collected by tissue collection device 140. In one application of the present invention, medical device 22, which is fluidly connected with suction manifold 50 via tubing 212, may take the form of a morcellation device.

Medical devices 24-26, which are shown fluidly connected with suction manifold 50 via respective tubing 202 may include, but are not limited to, a scope or instrument having a fluid outflow port (e.g., a hysteroscope), a buttocks drape having a suction port, and a floor fluid suction device.

Suction canister 30 may take the form of the first of multiple daisy-chained suction canisters. The suction canisters are coupled to a suction or vacuum source (not shown), such as those commonly found in a surgical suite. Adjustments to the suction levels associated with each medical device 22-28 may be carried out using standard multiple position pinch clamps, as shown in FIG. 1. In the embodiment shown in FIG. 4, cams 188a-188d may be used as the means for adjusting respective suction levels.

It should be appreciated that the fittings shown herein are for illustration purposes only and are not to be construed as limiting the present invention. In this regard, it is contemplated that the type of fittings used in the present invention may include, but are not limited to: hose fitting, barbed fitting, glue fitting, standard male (Luer) conical lock fitting, female (Luer) conical lock fitting, stepped fitting, threaded fitting, and proprietary or industry-standard quick disconnects, such as cam and groove type disconnects.

Other modifications and alterations will occur to others upon their reading and understanding of the specification. It is intended that all such modifications and alterations be included insofar as they come within the scope of the invention as claimed or the equivalents thereof.

Having described the invention, the following is claimed:

1. A suction manifold assembly comprising:
a suction manifold including:
a body defining an inner region;
a plurality of inlet ports, each inlet port having a passageway in fluid communication with the inner region and a fitting adapted for connection with a hose, wherein said plurality of inlet ports include a removable inlet port having a tissue collection device; and
an outlet port having a passageway in fluid communication with the inner region and a fitting adapted for connection with a hose.

2. A suction manifold assembly according to claim 1, wherein said tissue collection device includes a tissue collection chamber for collecting excised tissue, said tissue collection device allowing fluid to pass therethrough.

3. A suction manifold assembly according to claim 2, wherein said tissue collection device is a straining element.

4. A suction manifold assembly according to claim 3, wherein said straining element is comprised of at least one of the following: a mesh, a filter material, a screen, or a perforated media.

5. A suction manifold assembly according to claim 1, wherein said suction manifold assembly further comprises a mounting member dimensioned to receive said suction manifold.

6. A suction manifold assembly according to claim 1, wherein said mounting member includes a mounting bracket.

7. A suction manifold assembly according to claim 6, wherein mounting member further comprises a mounting plate having a plurality of support members for supporting one or more hoses connected to said plurality of inlet ports.

8. A suction manifold assembly according to claim 7, wherein mounting plate further includes a plurality of rotatable cams for engaging hoses connected to said plurality of inlet ports for regulating a suction level therethrough.

9. A suction manifold assembly according to claim 1, wherein said fittings are selecting from the following group: hose fitting, barbed fitting, glue fitting, standard male (Luer) conical lock fitting, female (Luer) conical lock fitting, stepped fitting, threaded fitting, and proprietary or industry-standard quick disconnects.

10. A suction manifold assembly according to claim 1, wherein the passageway of each inlet port has an inner diameter, wherein the inner diameter of at least one passageway is smaller than the inner diameter of at least one other passageway.

11. A suction manifold for receiving fluid from a plurality of fluid sources, said suction manifold comprising:
a body defining an inner region;
a plurality of inlet ports, each inlet port having a passageway in fluid communication with the inner region and a fitting adapted for connection with a hose, wherein said plurality of inlet ports include a removable inlet port having a tissue collection device for collecting excised tissue, said tissue collection device allowing fluid to pass therethrough; and
an outlet port having a passageway in fluid communication with the inner region and a fitting adapted for connection with a hose.

12. A suction manifold according to claim 11, wherein said tissue collection device is a straining element.

13. A suction manifold according to claim 12, wherein said straining element is comprised of at least one of the following: a mesh, a filter material, a screen, or a perforated media.

14. A suction manifold according to claim 11, wherein the passageway of each inlet port has an inner diameter, wherein the inner diameter of at least one passageway is smaller than the inner diameter of at least one other passageway.
